(19) United States
(12) Patent Application Publication
(10) Pub. No.: US 2009/0281634 A1
(43) Pub. Date: Nov. 12, 2009

(54) GUT PROSTHETIC

(75) Inventors: Thomas L. Abell, Ridgeland, MS (US); Christopher J. Lahr, Jackson, MS (US); Derek F. Lahr, Blacksburg, VA (US)

Correspondence Address: BUTLER, SNOW, O'MARA, STEVENS & CANNADA PLLC, 6075 POPLAR AVENUE, SUITE 500, MEMPHIS, TN, 38119, US

(73) Assignee: The University of Mississippi Medical Center Research Development Foundation, Jackson, MS (US)

(21) Appl. No.: 12/463,199

(22) Filed: May 8, 2009

Related U.S. Application Data
(60) Provisional application No. 61/051,575, filed on May 8, 2008.

(51) Int. Cl. A61F 2/04

(52) U.S. Cl. 623/23.65; 623/23.67

(57) ABSTRACT

A prosthetic device for the intestine is disclosed that bypasses an enterocutaneous fistula so that the intestinal tract can properly function without bowel contents leaking onto the skin. The prosthetic device includes means to securely connect and seal it to the intestine, means to push bowel contents through it, and means to prevent the backflow of bowel contents through the digestive tract.

### FIELD OF THE INVENTION

The present invention is generally directed toward accelerating the healing process after damage to the intestinal tract. More particularly, the invention is directed to an apparatus for treatment of an enterocutaneous fistula.

### BACKGROUND OF THE INVENTION

Stab wounds, gun shot wounds and surgical complications can result in an abnormal connection between the intestines and skin through which bowel contents may pass. This abnormal passage connecting the intestine to the skin is known as an enterocutaneous fistula (ECF). Ordinarily, these fistulas can be closed immediately after the stab wound, gunshot wound or surgical injury takes place without the occurrence of chronic ECF. However, sometimes associated injuries, severe life-threatening bleeding, or shock from blood loss or inadequate blood supply impair healing of intestinal wounds and ECF occurs many days or weeks after the initial injury.

Enterocutaneous fistulas present a major medical problem because they allow contents of the digestive system to leak out onto the skin. These small bowel contents include liquefied food as well as digestive enzymes and alkali which are caustic to the skin. Because the body produces large amounts of digestive fluids to digest food, more liquid escapes through the fistula than is ingested. Not only does the fistula result in damage to the skin and reduced gastrointestinal functionality, it also causes significant hygiene problems. The patient is unable to function normally until the drainage has been markedly reduced.

The occurrence of ECF weeks after initial injury may not be readily repaired surgically because of other life-threatening conditions such as inadequate blood clots, infection elsewhere in the body, or severe scar tissue or inflammation in the abdominal cavity. The marked inflammation and scarring that can occur after abdominal surgery can make any follow-up surgery difficult. The bowel wall can either become very thin and fragile, or it can become very thick and swollen. Either condition makes re-operation and immediate re-repair difficult, dangerous or impossible. Attempted re-operation can result in additional holes in the intestine which then must also be repaired. These holes may also break down, causing additional ECFs.

Currently, surgical repair of enterocutaneous fistula is a difficult procedure, involving a dangerous operation with the risk of causing additional fistulas. The current therapy is most simply described as keeping the patient alive until the body heals the fistula itself or until major surgery can be attempted.

Several steps may be taken to keep the patient alive and to reduce any discomfort. Total parenteral nutrition, the practice of providing nutrition in liquid form intravenously, could be used to bypass the normal digestive tract. This meets the patient's nutritional needs and thereby reduces the amount of fluid coming out of the ECF onto the skin.

Medication, such as somatostatin, may also be used to reduce bowel activity, resulting in lower ECF output. Additionally, the same apparatus used to cover colostomies could be used to cover the ECF openings to capture the ECF drainage to prevent skin damage and make life more comfortable for the patient. The wound can also be kept clean with the use of dressings.

The medical costs and financial burdens using the current enterocutaneous fistula treatments are huge. While the patient is in the hospital, costs can surpass $5,000 per day, and hospital stays may last for months. After leaving the hospital, most patients require total parenteral nutrition. This process can easily cost hundreds of dollars per day.

Furthermore, these measures do not properly address the release of intestinal fluids onto the skin. There is an important need for a device that can prevent the contents of the bowel from escaping through a fistula until the damage to the intestines can be properly treated.

### SUMMARY OF THE INVENTION

The present invention is a prosthetic device that may be inserted between the healthy ends of the intestine surrounding the enterocutaneous fistula, effectively bridging the discontinuity in the intestines. The prosthetic device would allow contents of the intestinal tract to pass through the bowels without leaking out. It would allow the patient to intake food normally and not require total parenteral nutrition for extended periods. The prosthetic device includes means for securing the device within the intestines and preventing digestive fluids from escaping from the intestinal tract. Additionally, the device may include means to simulate peristalsis thereby pushing bowel contents into the undamaged portion of the intestine. The prosthetic device may also include unidirectional valves to prevent contents from moving backward through the digestive tract.

Using the invention described herein, a patient suffering from an enterocutaneous fistula may recover sooner without extensive hospital stays.

### BRIEF DESCRIPTION OF THE DRAWINGS

Further advantages of the invention will become apparent by reference to the detailed description of preferred embodiments when considered in conjunction with the drawings, which are not to scale, wherein like reference characters designate like or similar elements to the several drawings as follows:

FIG. 1 depicts an embodiment of the invention.

FIG. 2 depicts another view of an embodiment of the invention.

FIG. 3 depicts an embodiment of the invention including an expandable inner member.

FIG. 4 depicts another view of an embodiment of the invention including an expandable inner member

FIG. 5 depicts an embodiment of the invention including an expandable inner member and unidirectional valves.

FIG. 6 depicts another view of an embodiment of the invention including an expandable inner member and unidirectional valves.

### DETAILED DESCRIPTION OF THE PREFERRED EMBODIMENT

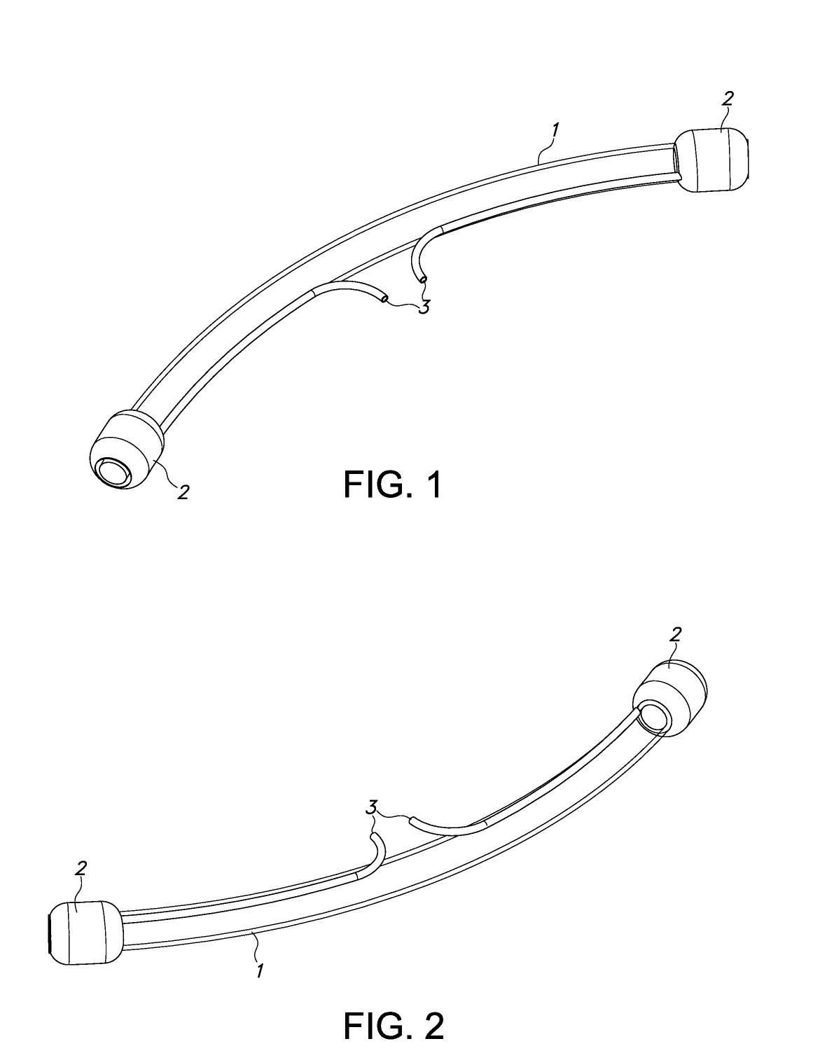

In one embodiment of the invention, depicted in FIG. 1 and FIG. 2, the prosthetic device consists of an elongated tube 1 which bridges both disconnected ends of the intestine. It includes means for securing the device into the small intestine and forming a seal such that bowel contents cannot seep around the prosthetic device. In the current embodiment, the seals are formed by means of inflatable cuffs 2 which encircle the elongated tube 1 and are located at both ends of the prosthetic device. The inflatable cuffs 2 may be inflated by passing air through inflation tubes 3 that may be connected to an air pressure source such as a syringe or air pump (not shown). The inflation tubes 3 may include valves that prevent air from escaping the inflatable cuffs 2.

Elongated tube 1 may be constructed of any biocompatible material. Preferably, the elongated tube 1 is constructed of a mildly deformable material such as silicone rubber latex, vinyl tubing, or a thermoplastic elastomer.

Tubing with an inflatable cuff, such as that found on Emergency Medicine Tubes manufactured by MALLICKRODT for insertion into the trachea, may be used at the ends of the prosthetic device. Such tubing consists of a vinyl tubing with inflatable cuffs and inflation tubes. Tubing with a single inflatable cuff has previously been disclosed in U.S. Pat. No. 4,387,711 to Merry. The tubing with the inflatable cuff may be connected to the elongated tube 1, or it may be one continuous piece. Pushing air or any gas into the inflatable cuffs 2 through inflation tubes 3 will result in expansion of the ends of the prosthetic device such that it forms a seal against the inner walls of the ends of the intestine. The seal prevents bowel contents from bypassing the prosthetic device. Other means to seal the prosthetic device against the inner walls of the intestine that are also known in the art may be used.

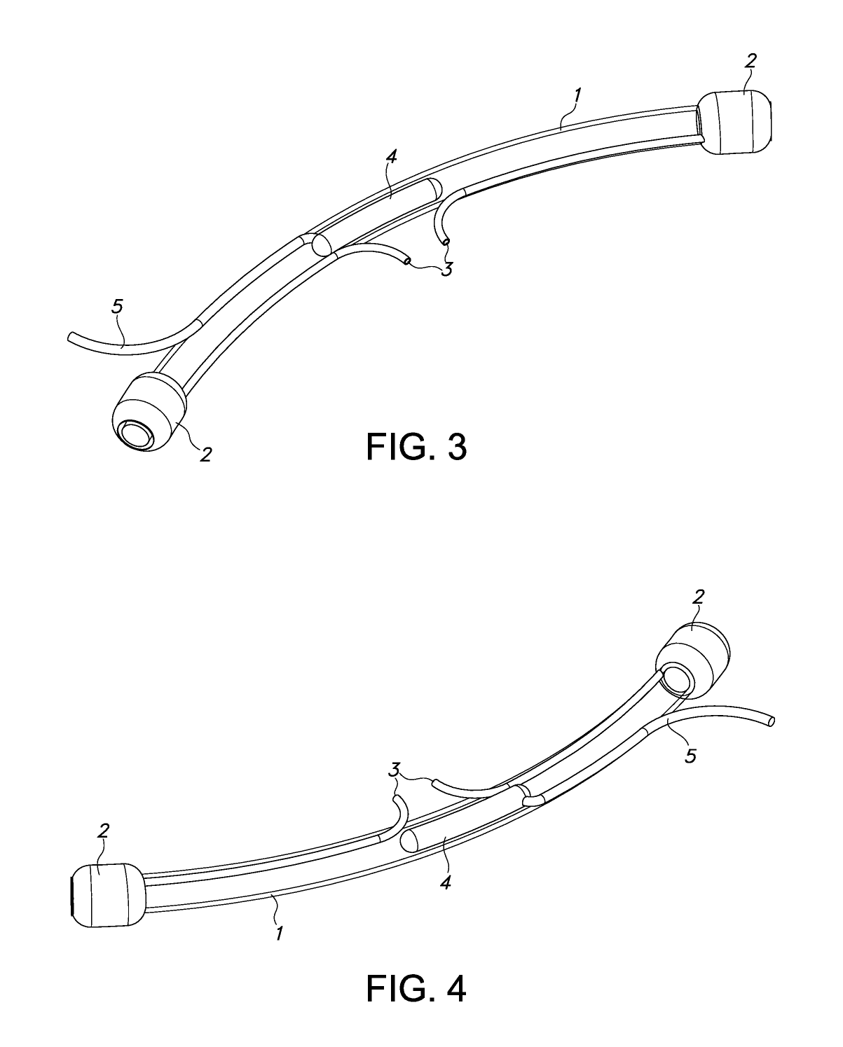

The claimed invention may optionally include means for promoting the motion of intestinal contents through the prosthetic device. In the pictured embodiment of FIG. 3. and FIG. 4, an expandable inner member 4 is present within the lumen of elongated tube 1. Inner member 4 is connected to an air pump via inner member inflation tube 5. This inner member inflation tube 5 passes through a small hole in the wall of elongated tube 1. The area around the hole may be sealed to prevent any intestinal fluids from escaping. The air pump can push air into or suction air from expandable inner member 4. Pushing air causes the expandable inner member 4 to inflate, expelling the bowel contents from the lumen of the prosthetic device.

The pump may then operate in reverse causing air to be suctioned out of the expandable inner member 4 such that the expandable inner member 4 contracts. The deflation and resulting contraction of the expandable inner member 4 allows more bowel contents to enter into the elongated tube 1 of the prosthetic device. Any pump that can alternate between pushing and pulling air may be used. Other means for mechanically moving the bowel contents in and out of the prosthetic device may be used, including, but not limited to, mechanical flaps or sequentially inflated balloons.

The expandable inner member 4 may be constructed of any deformable, non-corrosive material that can withstand the harsh acidic environment of the small intestine, such as plastic or preferably silicone. Inner member inflation tube 5 may be constructed of any narrow tubing such as vinyl tubing. In one embodiment, a balloon dilator manufactured my BOSTON SCIENTIFIC and sold under the MICROVASIVE CRE brand was used for both the inner member inflation tube 5 and the expandable inner member 4. This balloon dilator was inserted through a small hole in the elongated tube 1 such that the balloon acted as the expandable inner member. The elastic properties of the elongated tube 1 in our embodiment allowed it to stretch around the inner member inflation tube and form a seal.

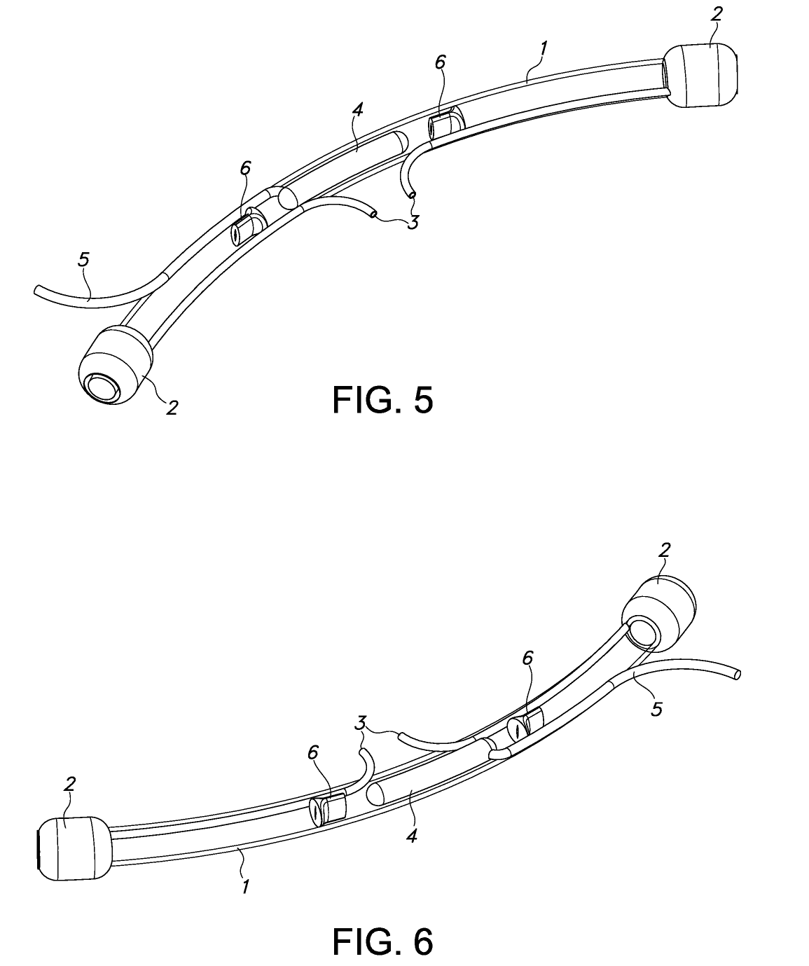

The prosthetic device may optionally include means for preventing the backflow of intestinal contents. As can be seen from FIG. 5 and FIG. 6, in the preferred embodiment, the invention includes unidirectional valves 6 that allow digestive contents to pass through the prosthetic device in one direction only. As the bowel contents are expelled from the lumen of the prosthetic device, the unidirectional valves 6 ensure that digestive contents are only moved toward the direction of the anus. In the present embodiment, the unidirectional valves 6 consist of a deformable material that opens to allow fluid to pass in one direction, but closes when fluid pressure builds in the opposite direction. Such valves are well known in the art. Other unidirectional valves are known in the art and may be used in the prosthetic device.

In practice, the surgeon positions the prosthetic device between the disconnected ends of the intestine, with attention to the direction of flow if the device contains unidirectional valves 6. The inflatable cuffs 2 are inserted far enough into each of the disconnected ends of the intestine such that their expansion will cause the inflatable cuffs to press against the undamaged intestinal wall. A syringe or other source of air, such as a pump, is connected to each inflation tube 3, and air is then forced into the inflatable cuff 2 until the inflatable cuff 2 presses firmly against the intestinal wall. Typically, the device is left in the patient until other major trauma heals.

If the prosthetic device contains an expandable inner member 4, to assist in the movement of digestive contents through the prosthetic device, then an inner member inflation tube 5 would be connected to an air pump capable of alternating between pushing or suctioning air.

When the patient has sufficiently recovered to allow for surgical reattachment of the two disconnected ends of the intestine, the surgeon will deflate the inflatable cuffs 2 by means of a pump capable of removing air, such as a syringe. The prosthetic device can then easily be removed from the patient.

It should be understood that features of any of these embodiments may be used with another in a way that will now be understood in view of the foregoing disclosure. For example, any embodiment could work with or without the expandable inner member 4, or with or without the unidirectional valves 6.

Although the present invention has been described and illustrated with respect to at least one preferred embodiment and uses therefor, it is not to be so limited since modifications and changes can be made therein which are within the full-intended scope of the invention.

We claim:

1. A prosthetic device for damaged intestines comprising
(a) an elongated tube; and
(b) inflatable cuffs encircling both ends of said elongated tube, said cuffs being inflatable by air supplied thereto to engage the walls of the intestine.

2. The prosthetic device of claim 1 further comprising means to expel the contents of the lumen of the elongated tube.

3. The prosthetic device of claim 2, wherein said means comprises an expandable inner member.

4. The prosthetic device of claim 3 wherein said expandable inner member expands by inflation.

5. The prosthetic device of claim 2 further comprising means to force said content of the lumen to expel in the direction of the anus.

6. The prosthetic device of claim 2 further comprising unidirectional valves configured to allow said contents of the lumen to only expel toward the anus.

7. A prosthetic device for preventing the release of digestive contents from an enterocutaneous fistula comprising:
(a) an elongated tube;
(b) inflatable cuffs encircling both ends of said elongated tube, said cuffs being inflatable by air supplied thereto to engage the walls of the intestine; and
(c) an expandable inner member capable of expelling contents of said elongated tube as it expands.

8. A prosthetic device for damaged intestines comprising
(a) an elongated tube;
(b) inflatable cuffs encircling both ends of said elongated tube, said cuffs being inflatable by air supplied thereto to engage the walls of the intestine;
(c) an expandable inner member capable of expelling contents of the elongated tube as it expands; and
(d) unidirectional valves position within said elongated tube positioned to direct the contents of the elongated tube in one direction.

9. A method for preventing the release of digestive contents from damaged intestines comprising
(a) positioning a prosthetic device comprising an elongated tube and inflatable cuffs encircling both ends of said elongated tube, said inflatable cuffs configured to engage the intestinal walls when inflated; and
(b) inflating said inflatable cuffs.

10. The method of claim 9 further comprising the step of inflating an expandable inner member located within the elongated tube.
